(19) United States
(12) Patent Application Publication
(10) Pub. No.: US 2014/0288349 A1
(43) Pub. Date: Sep. 25, 2014

(54) THERAPEUTIC DEVICE FOR TREATING A PREDEFINED BODY PART OF A PATIENT WITH RAYS

(71) Applicants: Steffen Seeber, Heidelberg (DE); Klaus Schewiola, Heidelberg (DE)

(72) Inventors: Steffen Seeber, Heidelberg (DE); Klaus Schewiola, Heidelberg (DE)

(73) Assignee: Deutsches Krebsforschungszentrum, Heidelberg (DE)

(21) Appl. No.: 14/235,229

(22) PCT Filed: Jul. 27, 2012
(86) PCT No.: PCT/EP2012/064751

§ 371 Date: Apr. 9, 2014

(30) Foreign Application Priority Data
Jul. 28, 2011 (EP) 11175776.1

(51) Int. Cl. A61N 5/10

(52) U.S. Cl. CPC A61N 5/1045; A61N 5/1077; A61N 5/1067; A61N 5/107; A61N 2005/1074; A61N 2005/1095; A61N 2005/1087; A61N 2005/109
USPC 600/1

(57) ABSTRACT

A therapeutic device (110) and a method for treating a predefined body part (112) of a patient with rays (116) are disclosed. The therapeutic device (110) has at least one ray source (118) for generating the rays (116). The therapeutic device (110) further has at least one collimator (120) for collimating and shaping the rays (116). The therapeutic device (110) further has at least one ray positioning system (122) for adjusting the position and direction of irradiating the rays (116) onto the patient. The therapeutic device (110) further has at least one patient positioning system (124) for positioning and orienting the patient. The therapeutic device (110) further comprises a control device (126). The control device (126) controls at least the collimator (120), the ray positioning system (122) and the patient positioning system (124). The control device (126) is a real-time system (128).

### TECHNICAL FIELD OF THE INVENTION

The invention relates to a therapeutic device for treating a predefined body part of a patient with rays, a method for controlling the therapeutic device and a use of a real-time system. The devices and methods according to the present invention specifically may be used in the field of cancer-treatment. However, other applications are possible.

### RELATED ART

Linear particle accelerators, such as so-called “linacs”, are the devices most commonly used for a therapeutic device for treating a predefined body part of a patient with rays, e.g. for external beam radiation treatments for patients with cancer. New treatment techniques and/or therapeutic devices may require more precision and/or versatility efficiency and/or reliability and/or dynamic behavior.

For defining the body part to be exposed to the radiation treatment, several devices are known for collimating the rays. Thus, so-called multi-leaf collimators are widely used for shielding the rays from a selected area and for defining an area of treatment. Examples of multi-leaf collimators for radiation treatment are disclosed in U.S. Pat. No. 4,794,629, US 2010/0278310 A1, U.S. Pat. No. 7,242,750 B2, US 2008/0191583 A1, US 2009/0041199 A1.

Therapeutic devices, e.g. as commonly used, in particular linac radio therapy set-ups, e.g. linear particle accelerators, typically may contain the following subsystems: a linear particle accelerator handling system, e.g. a gantry system; a patient support system, e.g. a patient couch; a x-ray beam generation system; at least one static patient set-up aid, a control console; a treatment room. The communication between these different subsystems is needed to be secured as per medical standards. Functional safety of all subsystems and/or all components and/or all systems needs to be guaranteed.

US 2009/003975 A1 discloses a robotic treatment delivery system including a linear accelerator (LINAC), and a robotic arm coupled to the LINAC. The robotic arm is configured to move the LINAC along at least four rotational degrees of freedom and one substantially linear degree of freedom.

Further, WO 2007/0141104 A2 provides a system and method of evaluating dose delivered by a radiation therapy system using a marker that indicates motion. The marker is associated with the patient. The motions (as well as operations) of the components and mechanisms of a treatment system can be controlled with a plurality of computers and/or controllers. Alternatively, a single system computer can be used to control the entire treatment system, which incorporates the processes and operations of all of the separate controllers and/or computers.

In US 2007/0007929 A1 a system and method of controlling power to a non-motor load are disclosed. A controller comprises two software layers: an application layer and a system layer. These two layers are developed independently and are integrated together by defining shared variables therebetween in accordance with the IEC61131-3 standard.

Common therapeutic devices for treating a predefined body part of a patient with rays, in particular, accelerator devices on the market, typically comprise a plurality of controlling units for controlling the various subsystems. Synchronization and control of all subsystems, e.g. subcomponents, mostly can not be established in real-time, particularly, with hard real-time requirements. Dynamic and 4D treatment methods are very often limited due to non-open standard solutions. Combinations with third party vendors usually are hardly possible. Thus, known therapeutic devices for treating a predefined body part of a patient with rays and known methods for controlling these devices and uses of them are disadvantageous and detrimental in several ways.

### PROBLEM TO BE SOLVED

It is therefore an objective of the present invention to provide a therapeutic device for treating a predefined body part of a patient with rays and a method for controlling this device and a use, which at least partially avoid the disadvantages of known devices, methods and uses. Specifically, a high-precise and dynamic treatment method with high time resolution and/or time constraints should be provided.

### SUMMARY OF THE INVENTION

This problem is solved by a therapeutic device for treating a predefined body part of a patient with rays, a method for controlling this device and a use according to the subject-matter of the independent claims. Preferred embodiments of the invention, which may be realized in an isolated way or in any arbitrary combination, are disclosed in the dependent claims.

As used in the present specification, the term “comprising” or grammatical variations thereof, such as the term “comprise” are to be taken to specify the presence of stated features, integers, steps or components or groups thereof, but do not preclude the presence or addition of one or more other features, integers, steps, components or groups thereof. The same applies to the term “having” or grammatical variations thereof, which is used as a synonym for the term “comprising”.

In a first aspect of the present invention, a therapeutic device for treating a predefined body part of a patient with rays is disclosed. The therapeutic device e.g. may be used for radiation therapy, preferably for cancer therapy. The predefined body part may be at least one organ or a part of an organ or at least a part of a tumor or a complete tumor and/or cancer cells. Preferably, the predefined body part may be at least a part of the skin and/or at least a part of the head and/or at least a part of the neck and/or at least a part of the breast and/or at least a part of the lung and/or at least a part of the prostate, e.g. a skin tumor and/or a tumor in the head and/or a tumor in the neck and/or a tumor in the breast and/or a tumor in the lung and/or a tumor in the prostate. The patient preferably may be a human being, e.g. an adult person or a child, but may also be an animal and/or a plant, preferably the patient may be a sick person and/or a person which may be treated.

The rays may be a beam, preferably a narrow beam of radiation, e.g. ionizing radiation, in particular a narrow beam of electromagnetic radiation, preferably suitable for cancer therapy.

The therapeutic device has at least one ray source for generating the rays. The ray source may preferably be a device for generating rays, e.g. a linear particle accelerator (linac) and/or another type of particle accelerator, e.g. a synchrotron, and/or a laser and/or the ray source may be a device which provides radiation, e.g. a device comprising at least one radioactive material. In a linear particle accelerator, e.g. a linac, electrons may be accelerated, e.g. by using a klystron, e.g. by using a complex magnet arrangement, a beam with about 6 to 30 MeV energy may be produced. The electrons may be used directly as a ray and/or the electrons may be collided with a target to generate, e.g. to produce, photons, e.g. high-energetic x-rays, preferably a beam of x-rays. The ray source preferably may comprise at least one x-ray beam generation system.

The therapeutic device further has at least one collimator for collimating and shaping the rays. The collimator preferably may be or may comprise one or more of: a multi-leaf collimator; an iris diaphragm collimator, such as described in WO 2006/119796; a pendular collimator, such as described in WO 03/043 698. However, alternatively or additionally, other collimators may be used. For potential embodiments of the multi-leaf collimator, reference may be made to the above-mentioned prior art documents. However, other embodiments of the collimator and/or of the multi-leaf collimator are feasible.

The rays may be formed by high-energy radiation beams. The collimator may be a device for collimating, e.g. increasing the coherence of the rays, and/or for shaping the rays, e.g. controlling a geometric shape, e.g. a diameter, and/or a direction of the rays. The collimator, preferably the multi-leaf collimator, may comprise at least one leaf-drive, e.g. with at least one set, preferably two sets of displaceable leaves arranged side by side, e.g. facing each other in order to impress a high-energy beam, e.g. the rays, with the shape of an irregularly formed treatment object, e.g. the predefined body part and/or the tumor, e.g. by enabling each of the leaves to assume a position oriented along the shape of the treatment object. The leaves of the multi-leaf collimator may also be called “shutter blades” or “lamellae”. The multi-leaf collimator may also be called “contour collimator” since due to the positioning of the leaves, contours of treatment objects, for example tumors, may be recreated for each beam application, each of which may occur from a certain solid angle. This may be important in order to protect adjacent healthy tissue, e.g. positions next to a tumor, to the greatest extent possible. In the case of critical tissue such as nerves, this may be particularly necessary in order to preserve their functional capability. For example, the rays, preferably beams, may be collimated and shaped by the collimator, in such a way that the rays may have exactly the same shape as the predefined body part, preferably, the collimator, preferably the multi-leaf collimator, may collimate and shape the rays in such a way that also intensity modulated radiation therapy (IMRT) and three-dimensional radiation control e.g. for dynamic radiation therapy may be possible, e.g. for taking account for movements of the body part during the treatment, e.g. due to breathing and/or sneezing and/or gulping and/or other movements of the patient and/or the predefined body part and/or displacements of the body part due to effects caused by metabolism, e.g. a filling of the bladder of the patient may change the position of a tumor of the prostate.

The therapeutic device further has at least one ray positioning system, e.g. a beam positioning system, for adjusting the position and direction of irradiating the rays onto the patient. The ray positioning system may be at least one device for adjusting the position and/or the direction of irradiating the rays onto the patient. The ray positioning system may be at feast partially at least part of the collimator and/or may be at least partially at least part of a separate device. The ray positioning system may be integrated in the ray source, e.g. by at least one device able to change a velocity and/or a direction of particles creating the rays or forming the rays, e.g. of the electrons which interact with the target to generate the x-rays, in a way, such that the position and/or the direction of the rays, e.g. the electrons and/or the x-rays, may be adjusted. The term “adjusting” may also comprise a control of the position and/or the direction, e.g. a regulation of the position and/or the direction and/or a modulation of the position and/or a modulation of the direction. The ray positioning system preferably may comprise at least one linear particle accelerator handling system, e.g. at least one gantry system.

The therapeutic device further has at least one patient positioning system for positioning and orienting the patient. The patient positioning system may be at least one device for positioning and/or orienting the patient, e.g. a patient positioning system may comprise at least one actuator, e.g. at least one stepper motor and/or at least one DC motor and/or at least one translation stage, preferably to perform a motion in at least one direction, preferably in three directions, preferably in three orthogonal directions, and/or to perform at least one rotation around at least one, preferably around all, of these directions, preferably by controlling at least one rotation angle. The patient positioning system may be able to position and/or orient the patient before starting the therapy, e.g. for comfortable seating and/or positioning of the rays and/or during treating the predefined body part of the patient with the rays, e.g. for taking account for movements of the body part as explained above and/or after the treatment, e.g. for comfortable getting up and/or seating a next patient. The term “positioning” may comprise at least controlling and/or defining a positioning along at least one direction, preferably along three directions, orthogonal to each other. The patient positioning system may comprise at least one bed and/or at least one seat and/or at least one couch and/or at least one patient support system, e.g. a patient couch, and/or at least one static patient set-up aid. The term “orienting” may comprise controlling and/or adjusting at least one angle between an axis of the patient and/or of the bed of the patient and/or of the couch of the patient and the at least one direction and/or preferably of the three directions, which are preferably orthogonal to each other. The patient positioning system may at least partially be part of the ray positioning system and/or may support the ray positioning system to adjust the position and/or the direction of irradiating the rays onto the patient and/or the patient positioning system may adjust the position and/or the direction of irradiating the rays onto the patient.

The therapeutic device further comprises a control device, wherein the control device controls at least the collimator, the ray positioning system and the patient positioning system, wherein the control device is a real-time system. The control device further may control preferably simultaneously one or more of the following subsystems of the therapeutic device: the ray source; at least one accelerator handling system for controlling a particle accelerator, preferably a linear particle accelerator, e.g. the gantry system; at least one patient support system, e.g. a patient couch; at least one x-ray beam generation system; at least one static patient set-up aid; at least one control console; and at least one treatment room and/or other subsystems.

The term “control” as used herein, preferably may comprise the action of managing, commanding, directing or regulating the behavior of other devices or systems. Further, additionally, the control may comprise collecting and/or exchanging of information, preferably digital information and/or analog information, e.g. voltages and/or currents, e.g. between the listed subsystems of the therapeutic device and/or of other sub-systems like the collimator and/or the ray positioning system and/or the patient positioning system. The term “control”may also comprise addressing the subsystems of the therapeutic device with jobs, e.g. addressing the patient positioning system to change the positioning and/or orientation of the patient and/or addressing the collimator to change the collimation of the rays and/or to change the shaping of the rays and/or addressing the ray positioning system to adjust and/or to change the position and/or the direction of irradiating the rays onto the patient. Information may contain different system parameters, e.g. subsystem parameters like positions, e.g. positions of the patient and/or of the rays and/or of the body part, and/or intensities, e.g. ray intensities and/or an evolution of movements of the body part and/or of at least a part of the patient.

As used herein, a real-time system may be defined as an arbitrary system, in which the duration of an operation, e.g. a delay time and/or a system cycle and/or a working cycle, and/or a response time is predefined, such as to a pre-defined maximum duration. Real-time systems preferably must execute, e.g. the system cycle and/or the working cycle, within strict constraints, in particular strict time constraints. Real-time systems may be said to have failed preferably if, e.g. a system cycle and/or a working cycle, is not completed before a deadline, wherein the deadline may be relative to an event for a system to be defined as real-time, it preferably must meet its time constraints and/or deadlines. A real-time system and/or one or more deadlines may be classified as hard real-time or soft real-time. Soft real-time may comprise systems and/or deadlines wherein usually the deadlines will not be missed. The attribute hard real-time may classify deadlines and/or systems wherein a strict time deadline is guaranteed. Missing a deadline within a hard real-time system may be classified as a total system failure. The goal of a hard real-time system may be to ensure that all time deadlines may be met. A cycle may be a period of time, preferably a working cycle.

The real-time system preferably may be a programmable logic controller (PLC), which may also be known as SPS (German “Speicher-Programmierbare Steuerung”). The real-time system preferably may be a hard real-time system and/or the programmable logic controller may be a hard real-time system. A programmable logic controller according to the present invention preferably may be a digital computer, which may be designed for multiple input and output arrangements and/or which may be applicable for extended temperature ranges and/or which may provide immunity to electrical noise and/or to vibration and/or to another impact. Within a PLC, output results preferably must be produced in response to input conditions within a deadline, e.g. within a bounded time, e.g. within the working cycle and/or within the system cycle. The PLC preferably may comprise at least one built-in communication port, e.g. for at least one Ethernet connection. The PLC further may be able to communicate over a network, e.g. Ethernet, to at least one other system, e.g. to at least one of the subsystems and/or at least one computer, e.g. a PC and/or another calculator. The PLC may comprise at least one logic for a control loop, e.g. at least one PID controller. The PLC preferably may comprise at least one software, which preferably may run a working cycle, which may run within a strict and/or controllable and/or predefined and/or guaranteed cycle time, e.g. the time of a working cycle and/or of a system cycle, which preferably has to be met, wherein the working cycle may be repeated preferably continuously.

The real-time system, e.g. the PLC, may be a system according to the IEC 61131-3 standard. Preferably, the real-time system may be a system according to the EN 61131 standard, which is based on the international IEC 61131 standard. The EN 61131 standard may deal with basics of programmable controllers like PLCs. An object-oriented development for distributed controller may be EN 61499, which also may be guaranteed by the PLC. EN 61131-3 as well as IEC 1131 and/or IEC 61131 previously may be the only worldwide valid standards for programming languages for programmable controllers, e.g. PLC. The programming languages, particularly the programming languages for programming the PLC, may be chosen from: instruction list (IL); ladder diagram (LD); function block diagram (FBD); sequential function chart (SFC); and structured text (ST). Other types of programming languages may also be used for programming the real-time system, preferably the PLC, and also the use of combinations of different programming languages may be possible.

The collimator, the ray positioning system and the patient positioning system and/or the ray source each, independently from each other, may comprise at least one driving unit.

The driving unit may be a subsystem or a component of a subsystem of the therapeutic device, e.g. for driving at least one of the subsystems mentioned above, e.g. the collimator. The driving unit may be electrically connected to the control device. A driving unit may comprise any device to drive the collimator and/or the ray positioning system and/or the patient positioning system and/or the patient positioning system and/or the ray source. The term “driving” may comprise e.g. an energy transformation, e.g. by the driving unit, e.g. a transformation of electrical energy to motional energy, e.g. by a driving unit of the collimator and/or of the ray positioning system and/or of the patient positioning system. The driving unit may also comprise at least one voltage transformer and/or at least one signal transformer and/or at least one logical unit. The term “electrically connected” may comprise a possibility to exchange information between the control device and the driving unit. The driving unit and/or the control device may be part of at least one network, e.g. connected via e.g. Ethernet and/or Bluetooth. Preferably, the term “electrically connected” may comprise the possibility of electron flow between the driving unit and the control device. The driving unit and the control device may additionally or alternatively be photonically connected, e.g. by at least one optical fiber and/or at least one optical beam path. The driving unit and the control device may be connected e.g. by at least one photonic fiber and/or at least one beam path and/or at least one interface and/or at least one cable and/or by the possibility of transferring information, e.g. via electromagnetic waves, e.g. via radio frequency.

The driving units, or at least one driving unit or a group of driving units, may be connected to the control device by at least one real-time Ethernet connection, preferably by at least one hard real-time Ethernet connection. The real-time Ethernet connection and/or the hard real-time Ethernet connection may comprise at least one device e.g. for connecting the driving units, or at least one driving unit to the control device by fulfilling the conditions of real-time, e.g. soft real-time conditions, most preferably hard real-time conditions, as defined above. The real-time Ethernet connection and/or the hard real-time Ethernet connection may be defined to provide a guarantee of connection and/or service to consistently operate deterministically and correctly. The real-time Ethernet connection and/or the hard real-time Ethernet connection preferably may be a part of a real-time communication network. The real-time Ethernet connection and/or the hard real-time Ethernet connection may comprise a bus system, such as an EtherCAT (Ethernet for control automation technology) system and/or a Profinet system. The therapeutic device and/or the real-time system may comprise at least one bus system, e.g. a fieldbus system, and optionally at least one redundant bus system, e.g. a redundant field bus system, e.g. EtherCAT, e.g. as a backup system. EtherCAT in general is a special case of a fieldbus, preferably EtherCAT may be real-time, preferably hard real-time, capable. Since 1999 fieldbus systems, preferably for industrial applications, e.g. EtherCAT; are standardized worldwide by the EEC 61158 standard (“Digital data communication for measurement and control—Fieldbus for use in industrial control systems”). Fieldbus systems in general are specified in the IEC 61784-1 standard as Communication Profile Families (CPF). Newer real-time capable Ethernet-based fieldbus systems may be assorted in the IEC 61784-2 standard. Protocol suites may define further fieldbus systems. The EtherCAT system may be preferably an open high performance Ethernet-based fieldbus system. Preferably, EtherCAT and/or the EtherCAT system and/or the real-time Ethernet connection may be able to provide e.g. short data update times, preferably short cycle times and/or working cycles, preferably with low communication jitter, e.g. for synchronization purposes. For synchronization, a distributed clock mechanism may be applied, which preferably may lead to very low jitters, e.g. to jitters of significantly less than 1 μs. The bus system and/or the real-time system and/or the PLC and/or the control device and/or the EtherCAT system and/or Profinet may be able to compensate delay times of information and/or signals and/or communication of actual values and/or control parameters and/or target values, e.g. delay times caused by different distances and/or different lengths of cables of the different driving units to the control device and/or to the real-time system and/or to the PLC. The bus system, e.g. a network, may comprise at least one circuit and/or at least one junction and/or at least one node.

The therapeutic device may comprise a treatment room, e.g. a therapy chamber, for treating the predefined body part of the patient with the rays. The treatment room may have shield elements, preferably one or more shield elements, for preventing the rays from leaving the treatment room. The control device may be located outside the treatment room and wherein the driving units preferably may be located inside the treatment room.

The treatment room preferably may be a room, e.g. a chamber, which may be isolated, preferably isolated according to the rays. The shield element preferably may be a device for protecting a passage of the rays and/or for isolating, preferably a control room and/or the environment and/or the control device, from the rays and/or for blocking and/or reflecting and/or absorbing the rays. The shield elements may be divided into several single shield elements. In principal, also only one shield element may be used for preventing the rays from leaving the treatment room. The shield element may comprise at least one material being able to attenuate and/or reflect and/or absorb and/or block the rays. The shield element may comprise at least one material, which filters the rays and/or parts of the rays, which may be dangerous for other parts of the therapeutic device and/or for the environment and/or for customers, like nurses and/or doctors and/or other medical staff and/or other patients.

The shield element and/or the shield elements may comprise e.g. at least one optical filter and/or at least one beam dump and/or at least one wall and/or at least one sealing and/or at least on floor being non-transparent for the rays and/or at least one electrical conductor and/or at least one Faraday cage and/or at least one material, which blocks at least a part of the rays, e.g. lead. The shield element may comprise several layers, e.g. wherein each layer may be non-transparent to another part of the rays, e.g. for another frequency range of the rays. Preferably, the ray source and/or the collimator and/or the ray positioning system and/or the patient positioning system and/or the patient also may be located inside the treatment room.

The control device preferably may be located outside the treatment room but may also be located inside the treatment room. The treatment room preferably may be bounded by the shield elements and/or the shield element. Treating the predefined body part may comprise influencing the body part, e.g. the tumor, by the radiation, preferably destroying cancer cells and/or the tumor, e.g. by ionization and/or by decreasing the temperature of at least a part of the tumor cells and/or the tumor and/or the predefined body part.

Each driving unit may be adapted to control at least one control parameter. Each driving unit may be adapted to provide an actual value of the control parameter to the control device. Each driving unit may be adapted to set the control parameter to a target value provided by the control device. The control parameter and/or the actual value and/or the target value may comprise at least one physical and/or chemical parameters, e.g. chosen from: the position of the patient; a relative position of a patient to a reference position; a position and/or a relative position of the predefined body part of the patient; a ray position; a relative ray position; a position of at least one leaf of the collimator; at least one axis of the collimator; the movements of the patient and/or the predefined body part of the patient, preferably a velocity and/or a frequency of the movements of the patient and/or the movements of the predefined body part of the patient; an intensity of the rays and/or a frequency of the rays and/or a flow of the rays and/or a temperature of the rays and/or a coherence of the rays and/or the frequency of the breathing of the patient and/or the frequency of the heartbeat of the patient and/or the temperature of the treatment room and/or the temperature of the patient or at least the temperature of a part of the patient, like the skin of the patient and/or the predefined body part of the patient. The target value and/or the actual value and/or the control parameter may be a single value and/or at least one single value and/or a mathematical or physical function and/or trace and/or complex values and/or lists of values and/or columns of values.

The control parameter may be a parameter, preferably a physical and/or chemical parameter, which should be controlled during the therapy and/or before the therapy and/or after the therapy and/or modulated and/or regulated. Preferably, the control parameter may be chosen from the physical and/or chemical parameters listed above. However, other possibilities are feasible alternatively or additionally.

The actual value preferably may be a value of the control parameter at a certain time, preferably at a time within the working cycle, more preferably at a predefined time. The actual value may be a single value of the control parameter or may be a continuous signal recorded e.g. over a certain amount of time.

The target value preferably may be a value of the control parameter to which the actual value is to be adjusted. Preferably, the control, e.g. a regulation, may be performed by the optional PID controller and/or by another part of the PLC and/or by an external controller. The actual value may be a feedback value and the actual value preferably may be regulated to the target value, preferably by a feedback loop.

The control device may be adapted to provide static and/or dynamic target values to the driving units. The target value may be a value of the control parameter which may be constant during the whole radiation therapy and/or during a period of the radiation therapy, e.g. during one day and/or one week and/or one month and/or during one sitting. A dynamic target value may be a target value of a control parameter, which preferably may change during time, e.g. continuously and/or discontinuously, e.g. between different working cycles and/or system cycles and/or different periods and/or which may change according to the movements of the patient and/or the movements of the predefined body part, e.g. caused by breathing and/or coughing and/or sneezing and/or tremor. A dynamic target value preferably may be calculated out of at least one control parameter and/or at least one simulation. The dynamic target value may also be necessary for performing the radiation therapy for destroying e.g. a tumor and/or for, preferably simultaneously, not destroying healthy parts of the patient.

The control device may be adapted to generate dynamic target values for at least one control parameter. The dynamic target values may be generated by using at least one predefined algorithm, preferably the algorithm may be able to predict a time-development of at least one target value and/or a time development of at least one actual value and/or at least one time development of the movements of the patient and/or of the movements of the predefined body part, preferably by using known trajectories of the movements of the predefined body part and/or of the patient.

The time development of the target values may be useful and/or necessary because of changing conditions, like breathing and/or sneezing and/or coughing and/or tremor of the patient and/or because of optimizing the impact of the rays e.g. on a tumor instead of impacting at least one a healthy part of the patient, which otherwise may be destroyed, too. The predefined algorithm may be programmed by a programming language, as described above, or by other programming languages.

The predefined algorithm may comprise one or more additional algorithms. The algorithm may be used, at least partially, e.g. to predetermine the movements of the patient and/or the movements of the predefined body part of the patient, e.g. by using at least one actual value and/or at least one calibration value. The predefined algorithm may be able to calculate a performance of the therapeutic device, e.g. variations of ray intensities and/or variations of ray positions, for destroying, e.g. the tumor and/or for protecting healthy parts of the patient. Known trajectories of the movements of the body part may be the breathing frequency and/or the heartbeat and/or a tracing comprising several positions. The patient may have position calibration marks, e.g. marked on the skin, e.g. by using at least one pencil, preferably for providing known trajectories of the movements of the predefined body part and/or of the patient.

The trajectories of the movements may be recorded by at least one motion control system. The trajectories of the movement may be recorded e.g. by visualization of the marks, e.g. by taking at least one picture and/or acquiring at least one image, e.g. with at least one camera, and/or by continuously imaging the marks and/or comparing the marks by using at least one laser beam and/or at least one laser system. The camera and/or the laser system may comprise at least one driving unit. The motion control system may e.g. comprise the camera and/or the laser system and/or the marks and/or the respective driving units. The acquired images, e.g. pictures, may be evaluated e.g. by the PLC and/or another calculator and/or by the algorithm. Preferably at least one control parameter may be determined by evaluating the acquired images, e.g. the move of the patient and/or the move of the predefined body part. Alternatively or additionally, position identifiers may be implemented into the patient, preferably into and/or on and/or next to the predefined body part, to get the trajectories and/or another control parameter. The identifiers may be at least one, preferably one to five, most preferably three, coils and/or identifiers comprising a material with less density for x-rays and/or comprising metallic and/or conducting materials. The coil and/or the identifier may be detected by a detector, e.g. a metal detector and/or an x-ray equipment for radiography and/or an magnetic resonance imaging device and/or another detector. The detector may comprise at least one driving unit. The detector and/or the camera preferably may be a subsystem of the therapeutic device. The trajectories may be relative distances compared to a fixed position, like a part of the treatment room, e.g. the table and/or the ray source and/or the trajectories may be distances. Preferably trajectories may be at least one trace.

The working cycle may be defined, wherein, during one working cycle, all actual values of the control parameters may be provided to the control device. Alternatively, during the working cycle, also only a predefined amount of the actual values of the control parameters may be provided to the control device. During one working cycle, target values for all control parameters, at least for one control parameter, may be provided to the driving units, preferably to at least one predefined driving unit, preferably by the control device. The control device may be adapted such that the working cycle may have a cycle time of no more than 100 μs, or even no more than 10 μs. Preferably, the cycle time may be smaller than typical time scales in which the predefined body part and/or the patient may move significantly, e.g. at least a distance of the diameter of the predefined body part, preferably at least a distance of 10% of the diameter of the predefined body part, most preferably at least a distance of 1% of the diameter of the predefined body part. Preferably, the cycle time may define the deadline, which may be guaranteed by the real-time system, preferably by the hard real-time system, most preferably by the PLC.

The control device may be adapted to provide a system clock for the therapeutic device. The system clock may be a clock and/or a device, which may be able to provide a clock pulse and/or a beat, preferably a periodic signal, preferably with high accuracy and periodicity. The clock pulse and/or the beat and/or the system clock may be generated in the control device or may be generated, e.g. by an atomic clock outside the therapeutic device. Inside the therapeutic device, the clock pulse and/or the beat and/or the system clock may be provided by a crystal oscillator, preferably a crystal oscillator which may be able to create an electrical signal, e.g. the beat, with a very precise frequency, e.g. for providing beats with frequencies from about 1 kHz to 100 MHz, specifically from 1 MHz to 50 MHz. The system clock may be triggering and/or driving and/or synchronize the working cycle and/or the system cycle and/or system cycles of at least one subcomponent and/or may define and/or measure the cycle time.

The collimator, the ray positioning system and the patient positioning system may be adapted to communicate with the control device in predefined time intervals, preferably defined by the system clock. Additionally, also the ray source and/or other subsystems of the therapeutic device may be adapted to communicate with the control device in predefined time intervals, preferably defined by the system clock. The communication preferably may be provided by Ethernet, preferably by real-time Ethernet, more preferably by hard real-time Ethernet. The predefined time interval may be periods of time, preferably periodic time intervals, e.g. synchronized with the working cycle. The predefined time intervals may be different for the different subsystems of the therapeutic device, like the collimator and/or the ray positioning system and/or the patient positioning system and/or the ray source, or may be the same for every subsystem of the therapeutic device. The predefined time intervals preferably may be shorter than the cycle time. The term “designed” by the system clock may comprise that time intervals may be synchronized with the system clock and/or the working cycle and/or that the time intervals may be triggered by the system clock.

The therapeutic device in total may have at least 100 control parameters, preferably 100 to 1000 control parameters and more preferably 150 to 220 control parameters. For example, about 160 control parameters may be related to leaves of the collimator and/or about 20 control parameters may be related to movable access of the therapeutic device, e.g. the ray positioning system and/or the patient positioning system. In total, one may need for this example 180 control parameters, which may have to be regulated, preferably by the real-time system, e.g. the hard real-time system, most preferably by the PLC.

The rays may be selected from the group consisting of: x-rays; γ-rays; ion-rays; α-rays; β-rays; neutral particle rays; neutral atom rays; heavy ion rays; atom rays; cold atom rays; electron rays; positron rays; proton rays; visible light rays; photonic rays; charged particle rays; ionizing radiation; continuous wave laser beams; pulsed laser beams; hadron rays; lepton rays; molecular rays. The rays and/or beams consisting of particles, like ion-rays and/or α-rays and/or β-rays and/or atom rays may have different, preferably stable, most preferably predefined and/or adjustable temperatures and/or velocities. Also other kinds of radiation and/or beams may be used, e.g. also combinations of different rays may be possible.

The collimator may have at least 100 leaves for blocking the rays, preferably at least 130 leaves and more preferably at least 160 leaves. The leaves may be individually positionable and/or controllable by individual actuators. As used herein, the term actuator refers to an arbitrary device adapted for mechanically moving and/or positioning an element or group of elements. Thus, the actuator may be selected from the group consisting of a mechanical drive, a piezoelectric actuator and a motor, specifically a stepper motor and/or a DC motor.

The ray positioning system and the patient positioning system, without the collimator, may comprise in total at least 10 axes, preferably at least 15 axes and more preferably at least 20 axes, wherein each axis may be separately controlled. As used herein the term “axis” may refer to a linear or nonlinear direction of movement of an object and/or to a rotational axis, around which an object may be rotated. Thus, the patient and/or the bed may be moved along and/or around at least one axis, preferably 6 axes. The collimator, preferably the multi-leaf collimator, may be established by using preferably one axis per leaf, e.g. about 80 leaves and 80 axis, preferably about 160 leaves and 160 axes.

The control device, e.g. the real-time system and/or the hard real-time system and/or the PLC, may be adapted to perform the role of a master device. The collimator, the ray positioning system and the patient positioning system and preferably also the ray source and/or other subsystems of the therapeutic device may be adapted to perform the role of a slave device. The role of a master device may comprise the control and/or command over one or more other devices and/or subsystems, e.g. at least one slave device. The role of a slave device may comprise execution of at least one command given by the master device. The device performing the role of a master device preferably may be connected, preferably for information exchange, with the device, which may be adapted to perform the role of the slave device. Preferably the controlling of the therapeutic device, including all subsystems, only may be performed by the control device, preferably by the real-time system, more preferably by the hard real-time system, e.g. by the PLC, preferably centrally controlled. Preferably the control device and/or the real-time system and/or the hard real-time system may be the only master device of the therapeutic device and/or the leading master device of the therapeutic device.

The real-time system, preferably the programmable logic controller, most preferably the hard real-time system, may be connected with at least one standard personal computer (PC) and/or at least one industrial PC (IPC).

In a further aspect of the present invention, a method for controlling a therapeutic device for treating a predefined body part of a patient with rays is disclosed. Preferably, reference may be made to the therapeutic device as disclosed above. Thus, the method may comprise the use of a therapeutic device according to the present invention, such as disclosed in the above-mentioned embodiments. The therapeutic device as used in the method has at least one ray source for generating the rays. The therapeutic device further has at least one collimator for collimating and shaping the rays. The therapeutic device further has at least one ray positioning system for adjusting the position and direction of irradiating the rays onto the patient. The therapeutic device further has at least one patient positioning system for positioning and orienting the patient. The method comprises the use of a control device. The method comprises controlling at least the collimator, the ray positioning system and the patient positioning system by using the control device, wherein the control device is a real-time system, preferably a programmable logic controller. Preferably the controlling of the therapeutic device, including all subsystems, only may be performed by the control device, preferably by the real-time system, more preferably by the hard real-time system, e.g. by the PLC, preferably centrally controlled.

In a further aspect of the present invention, a use of a real-time system, as a control device for controlling at least one collimator, at least one ray positioning system and at least one patient positioning system of a therapeutic device for treating a predefined body part of a patient with rays and optionally additionally also at least one other subsystem of the therapeutic device. Reference may be made to the therapeutic device as disclosed above. The therapeutic device has at least one ray source for generating the rays. The therapeutic device further has the collimator for collimating and shaping the rays. The therapeutic device further has the ray positioning system for adjusting the position and direction of irradiating the rays onto the patient. The therapeutic device further has the patient positioning system for positioning and orienting the patient. Preferably, the real-time system and/or the hard real-time system and/or the PLC may be the only control device of the therapeutic device or at least the leading control device.

The therapeutic device, the method for controlling the therapeutic device and the use of the real-time system as a control device for controlling the at least one collimator, the at least one ray positioning system and the at least one patient positioning system of the therapeutic device and optionally additionally at least one of the other subsystems of the therapeutic device according to the present invention, provide a large number of advantages over known devices, methods and uses. The device, method and use of the present invention may be able to guarantee hard real-time capability. They may also provide a maintainability of only one main system, preferably the PLC and/or the real-time system and/or the hard real-time system, and no distributed specialized subsystems, e.g. different controllers, e.g. with different topology. A central numerically controlled positioning, e.g. the ray positioning system and/or the patient position system, may be established on the PC system. E.g., PLC and a motion control, e.g. the ray positioning system and/or the patient positioning system and/or the motion control system, may be combined in at least one, preferably one, standard PC and/or in at least one, preferably one, industrial PC (IPC).

The present invention may also cause a reduction of cabling, e.g. the therapeutic device, according to the present invention, may need fewer cables, e.g. electronic connections and/or communication connections, than known devices. The system and the components, preferably of the therapeutic device according to the present invention, may be programmed via industrial standard interfaces with a common language, e.g. provided via IEC 61131-3 standard.

The exchangeability of components, e.g. at least one subsystem of the therapeutic device, may be much easier than in known therapeutic devices. Preferably, an explaceability and/or exchangeability of used technology, e.g. obsolete technology, with newer one, i.e. more modern technology, may be very easy, preferably without total redesign of the therapeutic device.

As modern PLC controls commonly have computing capability for a high amount of axles, axle control may be established centrally and/or synchronization of axles may easily be possible. Preferably each axis may comprise and/or have assigned at least one axle. An axle may be connected e.g. mechanically to a motor. The axle may be adapted to perform a rotation around the respective axis and/or to perform a translation along the respective axis. This may allow more dynamic in the total system, preferably of the therapeutic device according to the present invention.

Common interfaces as OPC (object linking and embedding for process control) exist and may be comprised by the therapeutic device according to the present invention. Common interfaces as OPC may allow easy connectivity to other linear particle accelerator and/or control systems, as PLC, e.g. for visualization and/or service purposes.

The therapeutic device, according to the present invention, preferably may support and/or comprise real-time, preferably hard real-time, communication bus systems, at least one real-time, preferably hard real-time, communication bus system, e.g. at least one bus system, most preferably at least one real-time communication bus system, e.g. based on Ethernet basis, e.g. EtherCAT.

A separation of the real-time system, e.g. a PLC host controller, e.g. the PLC, e.g. as master and/or as master device, and process closed clients may be possible. Process closed clients preferably may provide subsystem functionality, e.g. a process closed client may be a subsystem of the therapeutic device and/or a slave device and/or a driving unit. E.g, the separation may be caused by a location of the real-time system outside the treatment room and/or a location of the process closed clients inside the treatment room. The therapeutic device and/or the method and/or the use according to the present invention may establish more fail safe functionality, e.g. as main control, e.g. the control device, may be located outside the radiation area, e.g. outside the treatment room, preferably more fail safe functionality me be established as the real-time system and/or the PLC may be located outside the treatment room. The therapeutic device according to the present invention may provide very high reliability, e.g. due to the design of the real-time system, e.g. the PLC and/or terminals, e.g. the bus system and/or other subsystems of the therapeutic device. Preferably, the present invention may provide a meantime between failures (MTBF) of typically more than two years, or even more than five years, preferably of more than ten years. The therapy device and/or the method and/or the use according to the present invention may provide a very high safety integrity level. A capability may not be limited due to the application design, e.g. the PLC, but e.g. of PC capability, which may easily be planed for future redesigns. Preferably, the therapeutic device according to the present invention may provide a design which may allow high capability.

According to the present invention, preferably client functionality may be reduced, e.g. as the control device, e.g. the PLC, may provide already basic control functionalities and/or standards, e.g. pure functionality as data acquisition, e.g. position determination of linear encoders and/or rotational encoders, and/or actuator outputs, e.g. as servo controllers. Additionally special adapted actuators and/or sensors, e.g. for the PLC and/or already implemented in the PLC, may be available and may reduce effort for individual programming and/or adjustments significantly. The therapeutic device, preferably the use of the real-time system, e.g. the PLC, may establish easier synchronization of different system function controllers and/or subsystem function controllers and/or subsystems, e.g. device controls and/or driving units, as described above.

The therapeutic device, preferably the use of the real-time system, e.g. the use of the PLC, may open flexibility of the dynamic treatment techniques within the whole system, e.g. within the therapeutic device. A movement and/or a move, e.g. of the ray positioning system and/or the patient positioning system, at e.g. a patient support and/or a gantry and/or the collimator, preferably the multi-leaf collimator (MLC), and/or different flat panel (FP) devices may be synchronized and/or controlled in real-time, preferably in hard real-time. A flat panel may be a movable x-ray detection unit. The flat panel may be used for verification of an applied dose and/or of the intensity of the rays and/or of the adjusted collimator, e.g. the adjusted multi-leaf collimator, alternatively also at least one x-ray film may be used for this purpose, as commonly used in the art.

Additionally, the present invention may open flexibility to open linear particle accelerator (linac) architecture for third party vendors, as tracking and gating devices via standard interfaces, e.g. via PLC and/or the real time communication bus system, preferably based on Ethernet basis. The usage of a wide range of industrial offered standard devices may be possible as, e.g. so called terminals. The therapeutic device according to the present invention may provide, e.g. by the PLC, a standard interface to standard needs of data acquisition, e.g. of sensors and/or controlling of actuators and/or other subsystems of the therapeutic device, e.g. to establish axle movements. As data, e.g. information and/or commands and/or control parameters and/or actual values and/or target values, according to the present invention, preferably may always be distributed in real-time, preferably in hard real-time, collision avoidance and/or collision prevention may easily be established.

The present invention, in particular, the implementation of the real-time communication bus system, e.g. based on Ethernet, may provide several advantages compared to known therapeutic devices, like e.g. a usage of standard cables and/or plugs and/or interfaces. Intelligent network controllers, e.g. device controls, may be available to allow complete protocol processing, which preferably may take place within hardware, and may thus be fully independent of a run-time of protocol stacks, e.g. a central processing unit (CPU) performance and/or a software implementation.

With the therapeutic device and/or the method and/or the use according to the present invention, preferably the use of the real-time communication bus system, preferably based on Ethernet, typically performance values as following may be reached:

an update time for 1000 inputs/outputs (IOs) may be e.g. from 1 μs to 1 ms, preferably from 10 μs to 100 μs and most preferably approximately 30 μs, preferably including 10 cycle time; preferably up to 1486 bytes of process data may be exchanged, preferably with a single Ethernet frame, which may be equivalent to almost 12,000 digital inputs and outputs; the transfer of this data quantity may e.g. only take from 1 μs to 1 ms, preferably from 10 μs to 500 μs and most preferably may only take 150 μs; a communication with 100 servo axes may be extremely fast, e.g. within every 1 μs to 1 ms, preferably within every 10 μs to 500 μs and most preferably within about every 100 μs, all axes and/or all driving units and/or all driving units of the collimator and/or of the ray positioning system and/or of the patient positioning system may be provided with command values and/or control data and/or target values and/or information and/or may report their actual position and/or status and/or information and/or actual value and/or control parameter, preferably every 1 μs to 1 ms, most preferably about every 100 μs all actual values and/or target values may be provided and/or the predefined algorithm may predict the time-development of at least one target value and/or the time development of at least one actual value and/or the time development of the movements of the patient and/or of the movements of the predefined body part; a distributed clock technology, e.g. the use of the system clock, may allow a synchronization, e.g. at least of a part of the axles, preferably of all axles, e.g. within a deviation of smaller than 1 ms, preferably within a deviation of smaller than 100 μs, most preferably within a deviation of smaller than 1 μs; preferably most protocols may support safety integrity level (SIL) 3.

### SHORT DESCRIPTION OF THE FIGURE

Further optional details and features of the present invention may be derived from the subsequent description of preferred embodiments, preferably in combination with the dependent claims. Therein, the respective features may be realized in an isolated way or in arbitrary combinations. The invention is not restricted to the preferred embodiments. One embodiment is depicted schematically in the FIGURE. Identical reference numbers in the FIGURES refer to identical elements or to elements having identical or similar functions or to elements corresponding to each other with regard to their functionality.

FIG. 1 shows a therapeutic device for treating a predefined body part of a patient with rays.

### EMBODIMENTS

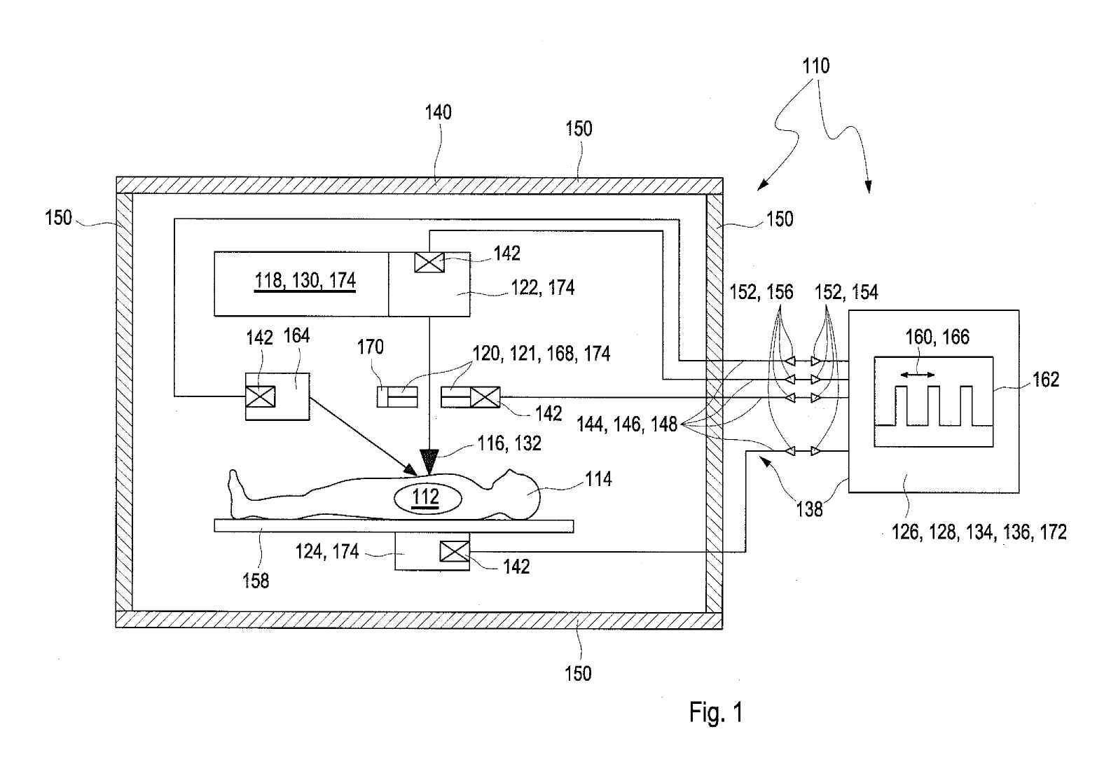

In FIG. 1, an embodiment of a therapeutic device 110 for treating a predefined body part 112 of a patient 114 with rays 116 is shown. The therapeutic device 110 has at least one ray source 118 for generating the rays 116. The therapeutic device 110 further has at least one collimator 120 for collimating and shaping the rays 116. The therapeutic device 110 further has at least one ray positioning system 122 for adjusting the position and direction of irradiating the rays 116 onto the patient 114. The therapeutic device 110 further has at least one patient positioning system 124 for positioning and orienting the patient 114. The therapeutic device 110 further comprises a control device 126. The control device 126 controls at least the collimator 120, the ray positioning system 122 and the patient positioning system 124. The control device 126 is a real-time system 128.

The collimator 120 may be or may comprise a multi-leaf collimator 121 and/or an iris diaphragm collimator, such as described in WO 2006/119796; a pendular collimator, such as described in WO 03/043 698; and/or another collimator.

The therapeutic device 110 may comprise at least one particle accelerator (linac) system and/or at least one particle accelerator system, such as a linear particle accelerator 130 and/or another beam generation device, which preferably may be used as ray source 118 or as a part thereof. The linear particle accelerator 130 may accelerate charged particles, e.g. ions and/or electrons. The accelerated electrons may be used for generating rays 116, e.g. x-rays 132. Preferably, the ray source 118 may be a linear particle accelerator 130. The linear particle accelerator 130 may be established by approximately 3 to 100, preferably by approximately 10 to 30 and most preferably by approximately 20 linear and/or rotational axles. The collimator 120, preferably the multi-leaf collimator (MLC) 121, additionally may be established by at least 100 axles, preferably by at least 130 axles and most preferably by at least 160 axles. The collimator 120 preferably may be a subsystem of the therapeutic device 110. The rays 116 may be preferably x-rays 132.

The real-time system 128 may be a programmable logic controller (PLC) 134. The real-time system 128, preferably the PLC 134, may be a hard real-time system 136. The therapeutic device 110 may provide a control of radiation therapy, e.g. a control of radiation therapy devices, e.g. of radiation therapy subsystems via PLC 134 technology. Preferably the PLC 134 may provide the control. Subsystems of the therapeutic device 110 may be the ray source 118 and/or the collimator 120 and/or the ray positioning system 122 and/or the patient positioning system 124 and/or the linear particle accelerator 130. The PLC 134 and/or the real-time system 128, e.g. a PLC control design and/or a real-time capable PLC network 138, may be used to control a radiation therapy system, preferably at least a subsystem of the therapeutic device 110, e.g. a radiation therapy device and/or its subsystems and/or at least one component, especially the collimator 120, e.g. the multi-leaf collimator 121 (MLC) device, e.g. to establish a platform for real-time synchronized system components, preferably for hard real-time synchronized system components, e.g. subsystems. The present invention may improve and/or may establish dynamic control and/or synchronization of all used axles in such a device, preferably in a radiation therapy device. The therapeutic device 110 according to the present invention may provide a different control approach of linear particle accelerators 130 and/or may provide control of its subsystems, especially on the multi-leaf collimator 121 and/or on the leaves 168 of the multi-leaf collimator 121 and/or on multi-leaf collimators 121. Preferably, at least one commercial standard PLC 134 and/or real-time communication technologies may be used for controlling the linear particle accelerator 130, preferably a medical linear particle accelerator device. A linear particle accelerator 130, and/or a linear particle accelerator radio therapy set-up and/or the therapeutic device 110 may comprise the following subsystems: at least one linear particle accelerator handling system, e.g. a gantry system; at least one patient support system, e.g. a patient couch and/or a bed; an x-ray beam generation system, e.g. a ray source 118; at least one static patient set-up aid; at least one control console, preferably the PLC 134; a treatment room, preferably a treatment room 140. The programmable logic controller (PLC) 134 and/or the real-time system 128 may be a computer system which may be typically used for automation of electromechanical processes, such as e.g. control of machinery and/or factory assembly lines. The PLC 134 may be a type of PLCs which may also be used in many industries and machines. Unlike general-purpose computers, PLCs may be designed for providing multiple input and output arrangements and/or extended temperature ranges and/or immunity to electrical noise and/or resistance to vibration and/or resistance to impact. Programs to control machine operation, e.g. programs to control devices 126 and/or components of the therapeutic device 110 may be typically stored in battery-baked and/or non-volatile memory. The PLC 134 may be a real-time control system, preferably since output results may be produced in response to input conditions within a bounded time and/or within a time span bordered by a borderline, preferably a deadline, otherwise, unintended operation may be a result. The PLC 134 may be programmed via standard-based programming languages. Preferably, the real-time system 128 may be a system according to the IEC 61131-3 standard. The standard-based programming languages may be defined under IEC 61131-3 standard. The real-time system 128 and/or the hard real-time system 136 and/or the PLC 134 and/or the PLC network 138 may be combined with a motion control system and/or a motion control and/or, e.g. comprising the collimator 120 and/or the ray source 118 and/or the ray positioning system 122 and/or the patient positioning system 124, e.g. in at least one standard PC or at least one industrial PC (IPC). The PLC 134 may include logic for at least one single-variable feedback analog control loop and/or at least one other control loop. Preferably, the PLC 134 may comprise at least one NC (numerical controller), e.g. at least one PID (“proportional, integral, derivative”) controller. Preferably, the therapeutic device 110 according to the present invention may establish be a system architecture with no more or less DCSs (distributed control systems), preferably, the therapeutic device 110 may be a therapeutic device 110 with no or as less as possible other control systems and/or control units and/or master devices 172 besides the PLC 134.

The collimator 120, the ray positioning system 122 and the patient positioning system 124 each may comprise at least one driving unit 142. The driving unit 142 may be electronically connected to the control device 126, preferably to the real-time system 128, and more preferably to the PLC 134. Alternatively, the ray source 118 and/or the collimator 120 and/or the ray positioning system 122 and/or the patient positioning system 124 and/or the linear particle accelerator 130 and/or the treatment room 140 and/or another subsystem of the therapeutic device 110 each may comprise at least one driving unit 142. The driving unit 142 may be electronically connected to the control device 126, preferably via cables 144.

The driving unit 142 or the driving units 142, preferably comprised by and/or attached to the collimator 120 and/or the ray positioning system 122 and/or the patient positioning system 124 and/or the ray source 118 and/or the multi-leaf collimator 121 and/or the linear particle accelerator 130 and/or the treatment room 140 and/or another subsystem and/or component of the therapeutic device 110, may be connected, preferably via the cables 144, to the control device 126 by at least one real-time Ethernet connection 146 and/or by at least one hard real-time Ethernet connection 148 and/or by at least one PLC network 138 and/or by at least one real-time communication technology and/or by at least one hard real-time communication technology and/or by at least one real-time bus and/or by at least one hard real-time bus and/or by PLC technology and/or by a real-time communication bus system, e.g. based on Ethernet, and/or by at least one hard real-time communication bus system, e.g. based on Ethernet. E.g., at least one driving unit 142 may be used for moving at least one gantry and/or the gantry system and/or the rays 116, e.g. for positioning, preferably around the patient 114.

The therapeutic device 110 may comprise at least one treatment room 140 for treating the predefined body part 112 of the patient 114 with the rays 116. The treatment room 140 may have at least one shield element 150 for preventing the rays 116 from leaving the treatment room 140. The shield element 150 and/or the shield elements 150 may comprise at least one treatment room shielding, e.g. at least one wall. The shield elements 150 and or the treatment room shielding and/or the walls may comprise at least one material which may be preferably at least partially, e.g. to 70% to 100%, preferably to 90% to 100%, most preferably to about 100%, non-transparent for at least a part of the rays 116, preferably for all the rays 116, e.g. for preventing rays 116 and/or radiation and/or radioactive particles from leaving the treatment room 140 and/or from reaching an area outside the treatment room 140, preferably for preventing the health of people being outside the treatment room 140 and/or for preventing disturbing electronics outside the treatment room 140, e.g. to prevent disturbing and/or perturbing the control device 126. Preferably, the shield element 150 may comprise at least one material comprising lead for preventing x-rays 132 from leaving the treatment room 140. The control device 126 preferably may be located outside the treatment room 140. The driving units 142 and/or at least a part of the driving units 142 may be located inside the treatment room 140.

Each driving unit 142 or at least a part of the driving units 142 may be adapted to control at least one control parameter 152. Each driving unit 142 or at least a part of the driving units 142 may be adapted to provide an actual value 154 of the control parameter 152 to the control device 126 and/or may be adapted to set the control parameter 152 to a target value 156 provided by the control device 126.

The control device 126 may be adapted to provide at least one static target value 156 and/or at least one dynamic target value 156 to at least one, preferably to all, driving units 142.

The control device 126 may be adapted to generate at least one target value 156, preferably at least one target value 156 for at least one control parameter 152. The dynamic target value 156 or the dynamic target values 156 may be generated preferably by using at least one predefined algorithm predicting the time-development of the target values 156, preferably by using at least one known trajectory of at least one movement of the body part 112 and/or of the patient 114. Dynamic target values 156 and/or the therapeutic device 110 according to the present invention may be used to improve dynamic treatment modes, e.g. as dynamic intensity modulated radiation therapy (IMRT) and/or adaptive tumor treatment and/or 4D treatment methods, e.g. as gating and/or tracking, preferably with high position precision and/or time-controlled precision. The control device 126 and/or the real-time system 128 and/or the PLC 134 may collect information, e.g. at least one actual value 154, e.g. at least one position and/or position information and/or at least one trajectory of the body part 112 and/or of the patient 114 and/or at least one position of the rays 116 and/or at least one position information of the rays 116 and/or at least one information of a position of the collimator 120 and/or at least one position and/or position information of at least one leaf 168 of the multi-leaf collimator 121 and/or at least an intensity of the rays 116 and/or at least one information about the linear particle accelerator 130. Preferably, the patient 114 may comprise at least one identifier and/or at least one mark, e.g. at least one coil, preferably one to five coils, most preferably three coils, preferably for determining the position and/or the position information and/or the trajectory of the body part 112 and/or the patient 114 and/or of a movement of the body part 112 and/or of the patient 114. The target value 156, e.g. the position and/or the position information of the body part 112 and/or the patient 114 and/or the rays 116 may be used to move and/or control the dynamic of a gantry, e.g. the ray positioning system 122 and/or a linear particle accelerator handling system, and/or the collimator 120, e.g. the multi-leaf collimator 121, and/or a table 158, e.g. the patient positioning system 124. Preferably, the table 158 may be comprised by the patient positioning system 124. The table 158 may be a bed and/or a couch and/or a seat and/or the bed may be able to be transformed, e.g. from a seat to a couch and/or the bed may be used for positioning of the patient 114 on it.

A working cycle 160 may be defined. During one working cycle 160, all or at least a part of the actual values 154 of the control parameters 152, may be provided to the control device 126 and/or target values 156 for all, or at least one, control parameters 152 may be provided to at least a part of the driving units 142, preferably to all of the driving units 142, by the control device 126. The control device 126 may be adapted such that the working cycle 160 may have a cycle time of no more than 100 μs or no more than 1 ms.

The control device 126 may be adapted to provide a system clock 162 for the therapeutic device 110. The system clock 162 may comprise and/or be connected to at least one electronic trigger and/or at least one crystal oscillator and/or at least one atomic clock. The electronic trigger and/or the crystal oscillator and/or the atomic clock may be implemented in the control device 126 or may be provided by an external device, e.g. by broadcasting a signal, e.g. by using at least one cable 144 and/or a radio frequency signal, to the control device 126. The collimator 120 and/or the ray positioning system 122 and/or the patient positioning system 124 and/or the ray source 118 and/or a motion control system 164, e.g. comprising at least one camera and/or at least one laser system, may be adapted to communicate with the control device 126 and/or the real-time system 128 and/or the PLC 134, preferably in predefined time intervals 166, preferably defined by the system clock 162, e.g. the working cycle 160.

The working cycle 160 and/or the time interval may be a time period in which all physical and/or chemical dimensions which should be regulated, e.g. all control parameters 152 and/or target values 156, may be recorded and/or target values 156 may be generated. The target value 156 may be a dynamic target value 156, e.g. for reacting on a movement of the patient 114 and/or of the body part 112. The movement of the body part 112 and/or of the patient 114, e.g. caused by breathing and/or coughing and/or sneezing and/or tremor, may be calculated by the PLC 134 and/or by a PC, e.g. by using the predefined algorithm. Preferably, within the working cycle 160 and/or the predefined time interval 166 known trajectories of movements of the body part 112 and/or of the patient 114 may be recorded, e.g. by using the motion control system 164, e.g. at least one camera and/or at least one laser system. The motion control system 164 may comprise the identifiers, e.g. coils, preferably one to five coils, most preferably three coils, preferably under the skin, e.g. in the predefined body part 112, and/or at least one other sensor and/or at least one mark, e.g. on the skin.

The therapeutic device 110 in total may have at least 100 control parameters 152, preferably 100 to 1000 control parameters 152 and more preferably 150 to 220 control parameters 152.

The rays 116 may be selected from the group consisting of: a narrow beam of electromagnetic radiation; light; ionizing radiation; charged particles; x-rays 132; γ-rays; ion-rays; α-rays; β-rays; neutron-rays; neutral atom rays; electron rays; proton rays; heavy ion rays; cold atom rays.

The collimator 120, preferably the multi-leaf collimator 121, may have at least 100 leaves 168 for blocking the rays 116, preferably at least 130 leaves 168 and more preferably at least 160 leaves 168. The leaves 168 may be individually positionable and/or controllable, e.g. by at least one individual actuator 170.

The ray positioning system 122 and/or the patient positioning system 124 without the collimator 120, preferably without the multi-leaf collimator 121, may comprise in total at least 10 axles and/or axes, preferably at least 15 axles and/or axes and more preferably at least 20 axles and/or axes. Preferably, each axle and/or axis may be controlled separately.

The control device 126 may be adapted to perform the role of a master device 172 or may be a master device 172. The collimator 120 and/or the ray positioning system 122 and/or the patient positioning system 124 may be adapted to perform the role of a slave device 174 and/or may be slave devices 174. The ray source 118 also may perform the role of a slave device 174 or may be a slave device 174.

Preferably, the control device 126 and/or the real-time system 128 and/or the PLC 134 may be used for controlling subsystems, e.g. slave devices 174, preferably all subsystems, e.g. the ray source 118, preferably the linear particle accelerator 130. Preferably, the real-time system 128 may comprise at least one real-time protocol and/or at least one real-time bus. Preferably, all subsystems of the therapeutic device 110 may be controlled by the control device 126 and/or by the real-time system 128 and/or by the PLC 134. In an alternative embodiment of the present invention, only a part of the subsystems of the therapeutic device 110 may be controlled by the control device 126 and/or by the real-time system 128 and/or by the PLC 134, e.g. only the collimator 120, e.g. the multi-leaf collimator 121, may be controlled by the control device 126 and/or by the real-time system 128 and/or by the PLC 134.

A computing capacity of the PC may enable axis and/or axle motion simultaneously with the PLC 134, whereby a position control system and/or a position controller and/or the motion control and/or the motion control system 164 may be used and/or may usually be provided simultaneously with at least one calculation, e.g. the simulation and/or the target value 156, by and/or from the PC and/or the PLC 134 and/or the control device 126. The computing capacity of the PC may enable many axes and/or axles to be positioned simultaneously.

A pure functionality of all axles or at least a part of the axles may be generalized as linear and/or rotational actuators 170 and/or according sensors. Preferably, a double amount of sensor systems and/or a double amount of the subsystems and/or a double amount of the control device 126 and/or a double amount of the real-time system 128 and/or a double amount of the PLC 134 may be used for redundancy and/or verification purposes. Instead of double amounts also more systems and/or more duplicates may be comprised by the therapeutic device 110, e.g. for safety purposes.

In another aspect of the present invention, a method for controlling the therapeutic device 110, e.g. as described above and as shown in FIG. 1, for treating the predefined body part 112 of the patient 114 with rays 116 may be explained by FIG. 1. The therapeutic device 110 has at least one ray source 118 for generating the rays 116. The therapeutic device 110 further has at least one collimator 120 for collimating and shaping the rays 116. The therapeutic device 110 further has at least one ray positioning system 122 for adjusting the position and direction of irradiating the rays 116 on the patient 114. The therapeutic device 110 further has at least one patient positioning system 124 for positioning and orienting the patient 114, wherein the method comprises the use of a control device 126. The method comprises controlling at least the collimator 120, the ray positioning system 122 and the patient positioning system 124 by using the control device 126. The control device 126 is a real-time system 128.

In another aspect of the present invention, a use of a real-time system 128, preferably as described above and by FIG. 1, as a control device 126 for controlling the at least one collimator 120, at least by a ray positioning system 122 and at least one patient positioning system 124 of the therapeutic device 110, preferably as described above and by FIG. 1, for treating a predefined body part 112 of a patient 114 with rays 116. The therapeutic device 110 has at least one ray source 118 for generating the rays 116. The therapeutic device 110 further has the collimator 120 for collimating and shaping the rays 116. The therapeutic device 110 further has the ray positioning system 122 for adjusting the position and direction of irradiating the rays 116 on the patient 114. The therapeutic device 110 further has the patient positioning system 124 for positioning and orienting the patient 114.

### LIST OF REFERENCE NUMBERS

110 therapeutic device
112 body part
114 patient
116 rays
118 ray source
120 collimator
121 multi-leaf collimator
122 ray positioning system
124 patient positioning system
126 control device
128 real-time system
130 linear particle accelerator
132 x-rays
134 PLC
136 hard real-time system
138 PLC network
140 treatment room
142 driving unit
144 cables
146 real-time Ethernet connection
148 hard real-time Ethernet connection
150 shield element
152 control parameter
154 actual value
156 target value
158 table
160 working cycle
162 system clock
164 motion control system
166 predefined time interval
168 leaf
170 actuator
172 master device
174 slave device

1. A therapeutic device for treating a predefined body part of a patient with rays, the therapeutic device having at least one ray source for generating the rays, the therapeutic device further having at least one collimator for collimating and shaping the rays, the therapeutic device further having at least one ray positioning system for adjusting the position and direction of irradiating the rays onto the patient, the therapeutic device further having at least one patient positioning system for positioning and orienting the patient, wherein the therapeutic device further comprises a control device, wherein the control device controls at least the collimator, the ray positioning system and the patient positioning system, wherein the control device is a real-time system.

2. The therapeutic device according to claim 1, wherein the collimator is a multi-leaf collimator.

3. The therapeutic device according to claim 1, wherein the real-time system is a programmable logic controller.

4. The therapeutic device according to claim 1, wherein the real-time system is a system according to the IEC 61131-3 standard.

5. The therapeutic device according to claim 1, wherein the collimator, the ray positioning system and the patient positioning system each comprise at least one driving unit, wherein the driving units are electrically connected to the control device.

6. The therapeutic device according to claim 5, wherein the driving units are connected to the control device by at least one real-time Ethernet connection.

7. The therapeutic device according to claim 5, wherein the therapeutic device comprises a treatment room for treating the predefined body part of the patient with the rays, the treatment room having shield elements for preventing the rays from leaving the treatment room, wherein the control device is located outside the treatment room and wherein the driving units are located inside the treatment room.

8. The therapeutic device according to claim 1, wherein each driving unit is adapted to control at least one control parameter, wherein each driving unit is adapted to provide an actual value of the control parameter to the control device and to set the control parameter to a target value provided by the control device.

9. The therapeutic device according to claim 8, wherein the control device is adapted to provide one or both of static or dynamic target values to the driving units.

10. The therapeutic device according to claim 9, wherein the control device is adapted to generate dynamic target values for at least one control parameter, wherein the dynamic target values are generated by using a predefined algorithm predicting the time-development of the target values.

11. The therapeutic device according to claim 5, wherein a working cycle is defined, wherein, during one working cycle, all actual values of the control parameters are provided to the control device and target values for all control parameters are provided to the driving units by the control device, wherein the control device is adapted such that the working cycle has a cycle time of no more than 100 μs.

12. The therapeutic device according to claim 11, wherein the control device is adapted to provide a system clock for the therapeutic device, wherein the collimator, the ray positioning system and the patient positioning system are adapted to communicate with the control device in predefined time intervals defined by the system clock.

13. The therapeutic device according to claim 7, wherein the therapeutic device in total has at least 100 control parameters.

14. The therapeutic device according to claim 1, wherein the rays are selected from the group consisting of: x-rays; γ-rays; ion rays; α-rays; β-rays; neutron rays.

15. The therapeutic device according to claim 1, wherein the collimator has at least 100 leaves for blocking the rays, wherein the leaves are individually positionable and controllable by individual actuators.

16. The therapeutic device according to claim 1, wherein the ray positioning system and the patient positioning system comprise in total at least 10 axes, wherein each axis may be separately controlled.

17. The therapeutic device according to claim 1, wherein the control device is adapted to perform the role of a master device, wherein the collimator, the ray positioning system and the patient positioning system are adapted to perform the role of a slave device.

18. A method for controlling a therapeutic device for treating a predefined body part of a patient with rays, the therapeutic device having at least one ray source for generating the rays, the therapeutic device further having at least one collimator for collimating and shaping the rays, the therapeutic device further having at least one ray positioning system for adjusting the position and direction of irradiating the rays onto the patient, the therapeutic device further having at least one patient positioning system for positioning and orienting the patient, wherein the method comprises the use of a control device, wherein the method comprises controlling at least the collimator, the ray positioning system and the patient positioning system by using the control device, wherein the control device is a real-time system.

19. A method of controlling at least one collimator, at least one ray positioning system and at least one patient positioning system of a therapeutic device for treating a predefined body part of a patient with rays, the method using a real-time system as a control device, wherein the therapeutic device has at least one ray source for generating the rays, wherein the therapeutic device further has the collimator for collimating and shaping the rays, wherein the therapeutic device further has the ray positioning system for adjusting the position and direction of irradiating the rays onto the patient, wherein the therapeutic device further has the patient positioning system for positioning and orienting the patient.

20. The therapeutic device according to claim 10, wherein the dynamic target values are generated by using the predefined algorithm predicting the time-development of the target values, by using known trajectories of movements of the body part.

21. The therapeutic device according to claim 13, wherein the therapeutic device in total has 100 to 1000 control parameters.

22. The therapeutic device according to claim 13, wherein the therapeutic device in total has 150 to 220 control parameters.

23. The therapeutic device according to claim 15, wherein the collimator has at least 130 leaves.

24. The therapeutic device according to claim 15, wherein the collimator has at least 160 leaves.

25. The therapeutic device according to claim 16, wherein the ray positioning system and the patient positioning system comprise in total at least 15 axes.

26. The therapeutic device according to claim 16, wherein the ray positioning system and the patient positioning system comprise in total at least 20 axes.
